(19) United States
(12) Patent Application Publication
(10) Pub. No.: US 2024/0194331 A1
(43) Pub. Date: Jun. 13, 2024

(54) SYSTEM AND METHOD FOR MANAGEMENT AND DISTRIBUTION OF INVISIBLE DENTAL BRACES CONTAINING PRESSURE-SENSITIVE SENSORS

(71) Applicant: 3D GLOBAL BIOTECH INC., New Taipei City (TW)

(72) Inventor: Keng-Liang OU, Taipei City (TW)

(73) Assignee: 3D GLOBAL BIOTECH INC., New Taipei City (TW)

(21) Appl. No.: 18/532,852

(22) Filed: Dec. 7, 2023

Related U.S. Application Data
(60) Provisional application No. 63/431,325, filed on Dec. 9, 2022.

(51) Int. Cl. G16H 40/20; A61C 7/00; A61C 7/08; G16H 15/00; G16H 20/40; G16H 40/40; G16H 40/67; G16H 80/00

(52) U.S. Cl. CPC G16H 40/20; A61C 7/002; A61C 7/08; G16H 15/00; G16H 20/40; G16H 40/40; G16H 40/67; G16H 80/00; A61C 2204/007

(57) ABSTRACT

The invention is a system and method for the management and distribution of invisible dental braces containing pressure-sensitive sensors for use in dentistry. The system and method include the steps of: providing an invisible dental brace fixedly secured with at least one pressure-sensitive sensor and a chip, wherein said at least one pressure-sensitive sensor generates a sensor signal related to the wearing conditions; writing additional information, including the name of the orthodontic patient into the chip; applying the invisible dental brace to the teeth of the orthodontic patient; writing further information, including special conditions that occur during applying the invisible dental brace; transmitting of a data, including the sensor signal, additional information, and further information, etc. to a remote device; and processing the data by the remote device for the dentist to modify the prescribed plan of orthodontic treatment in a real-time manner based on the data.

### TECHNICAL FIELD

The present invention relates to the field of dental braces, and more particularly to the system and method for management and distribution of invisible dental braces containing pressure-sensitive sensors.

### BACKGROUND OF THE INVENTION

Teeth misalignment is very common in the population and is considered improper for medical or aesthetic reasons. Teeth misalignment is caused by improper teeth position and may result in other dental illnesses, such as tooth decay and periodontal diseases. To correct the teeth misalignment, orthodontic treatment attempts to properly align the teeth by applying physical pressure or force to the teeth through an orthodontic appliance, such as dental braces, shell aligners, and the like. The orthodontic appliances are configured to move one or more teeth from a misaligned position to a correct(target) position according to a prescribed plan of orthodontic treatment.

Recently, with the advance in the field of material engineering and rapid prototyping techniques, invisible dental braces have been developed and are widely accepted by orthodontic patients and dentists. The invisible dental braces are advantageous for reasons of being free of dietary restriction, invisible appearance, and being more comfortable and less pain.

However, the process of orthodontic treatment is complicated, and sometimes the orthodontic treatment is not as effective as expected. Therefore, there is a compelling need to provide the dentist with a system for monitoring the wearing condition of the invisible dental brace used by the orthodontic patient, which enables the dentist to adjust the prescribed plan of orthodontic treatment in a real-time manner according to the wearing condition. Consequently, inventors of the present application have made a tremendous effort to make inventive research and eventually provide a system and method for the management and distribution of invisible dental braces containing pressure-sensitive sensors to facilitate the dentists to modify the prescribed plan of orthodontic treatment in a real-time manner according to the wearing conditions.

### SUMMARY OF THE INVENTION

The prime objective of the present invention is to disclose a system and method for the management and distribution of invisible dental braces containing pressure-sensitive sensors for use in dentistry. The system and method include the steps of providing an invisible dental brace fixedly secured with at least one pressure-sensitive sensor and a chip, wherein said at least one pressure-sensitive sensor generates a sensor signal related to the wearing conditions, including the wearing time of the invisible dental brace by an orthodontic patient and a pressure applied by the teeth of the orthodontic patient to the invisible dental brace, and identification of each tooth; writing additional information, including the name of the orthodontic patient into the chip by a read-write device; applying the invisible dental brace to the teeth of the orthodontic patient; writing further information, including special conditions that occur during applying the invisible dental brace; transmitting of a data, including the sensor signal, additional information, and further information, etc. to a remote device; and processing the data by the remote device for the dentist to modify the prescribed plan of orthodontic treatment in a real-time manner based on the data and to order a new invisible dental brace according to a modified plan of orthodontic treatment.

For achieving the prime objective mentioned above, the present invention provides an embodiment of a method for the management and distribution of invisible dental braces containing pressure-sensitive sensors, comprising the following steps:
providing an invisible dental brace by a manufacturing and distribution facility to a dentist, wherein at least one pressure-sensitive sensor and a chip are fixedly secured onto the invisible dental brace, wherein said at least one pressure-sensitive sensor is configured to generate a sensor signal related to wearing time of the invisible dental brace and a pressure applied by teeth of an orthodontic patient, wherein an identification number regarding the invisible dental brace is written into the chip;
writing additional information, including the name of the orthodontic patient wearing the invisible dental brace and an initial time of wearing, into the chip by a read-write device provided by the manufacturing and distribution facility to the dentist;
applying the invisible dental brace to the teeth of the orthodontic patient by the dentist;
writing further information into the chip by the read-write device, including special conditions that occur during applying the invisible dental brace to the teeth of the orthodontic patient;
transmitting of a data, including the sensor signal, identification number, additional information, and further information to a remote device during the usage of the invisible dental brace by the patient; and
processing the data by a specifically designed software or application program of the remote device for the dentist to modify the prescribed plan of orthodontic treatment in a real-time manner based on the data, and based on a modified prescribed plan of orthodontic treatment, an order of a new invisible dental brace is delivered to the manufacturing and distribution facility to initiate a manufacture of the new invisible dental brace.

In addition, the chip contains a memory, wherein the memory can be an EEPROM (Electrically-Erasable Programmable Read-Only Memory) or a flash memory for storing information. The memory can be divided into two parts—one read-only part for storing encrypted read-only information and one read-write part. The read-only part of the memory is for storing information such as a discrete identification number assigned to the invisible dental brace. The read-write part of the chip is for storing the data, including sensor signals, additional information, and further information.

Furthermore, the data can be transmitted to a remote device through a wire or a wireless communication technology. Further, the chip contains a wireless communication circuit and the data can be transmitted to a remote device through any existing wireless communication technologies including Wi-Fi, Bluetooth, WiMax, and cellular network.

Moreover, said at least one pressure-sensitive sensor may be a force-sensitive sensor and comprises a force-sensitive film, a pressure-sensitive film, a resistive film, a capacitive film or a piezoresistive pressure sensor.

Besides, the remote device can be any computing device or system, such as a personal computer, laptop, tablet, mobile device, wearable device, etc.

Further, the remote device is installed with a specifically designed software or application program that can process the data into an understandable and visual chart and 3D images representing the current teeth configuration of the orthodontic patient for the dentist to adjust the prescribed plan of orthodontic treatment accordingly.

### BRIEF DESCRIPTION OF THE DRAWINGS

The invention, as well as a preferred mode of use and advantages thereof, will be best understood by referring to the following detailed description of an illustrative embodiment in conjunction with the accompanying drawings, wherein:

FIG. 1 shows an illustrative diagram of an invisible dental brace containing a pressure-sensitive sensor;

FIG. 2 shows an illustrative diagram of a system for the management and distribution of invisible dental braces containing pressure-sensitive sensors according to the present invention;

FIG. 3 shows a flowchart of a method for the management and distribution of invisible dental braces containing pressure-sensitive sensors according to the present invention; and

FIG. 4 shows a block diagram of a system for the management and distribution of invisible dental braces containing pressure-sensitive sensors according to the present invention.

### DETAILED DESCRIPTION OF THE PREFERRED EMBODIMENTS

To better illustrate the advantages of a system and method for the management and distribution of invisible dental braces containing pressure-sensitive sensors according to the present invention and its contributions to the art, preferred embodiments of the present invention will be described in detail concerning the attached drawings hereafter.

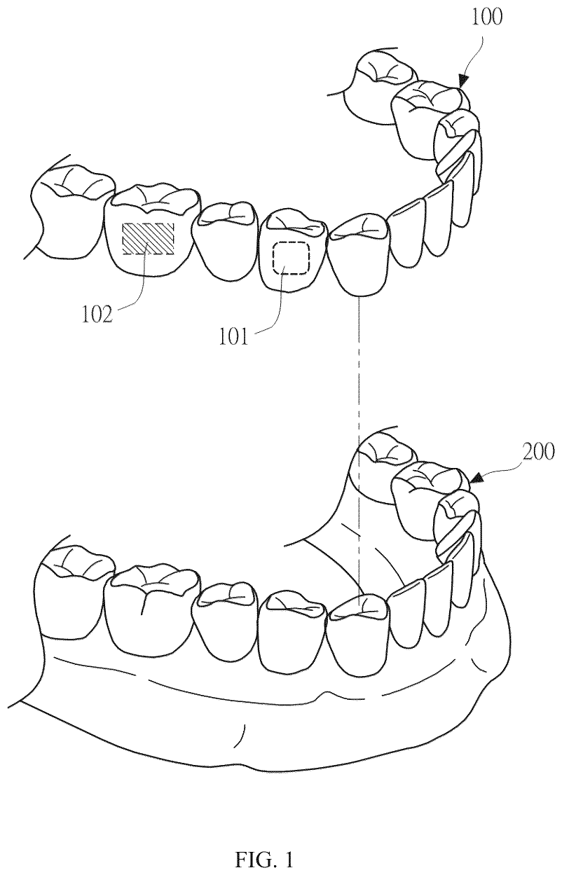

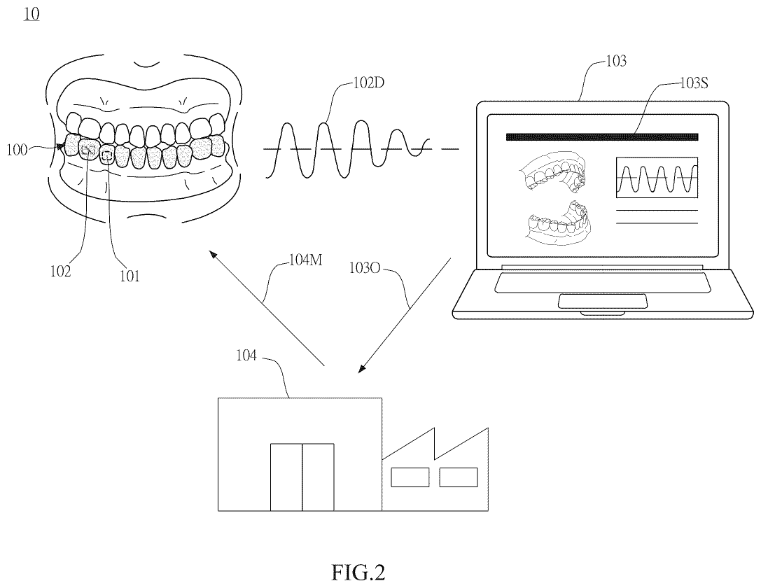

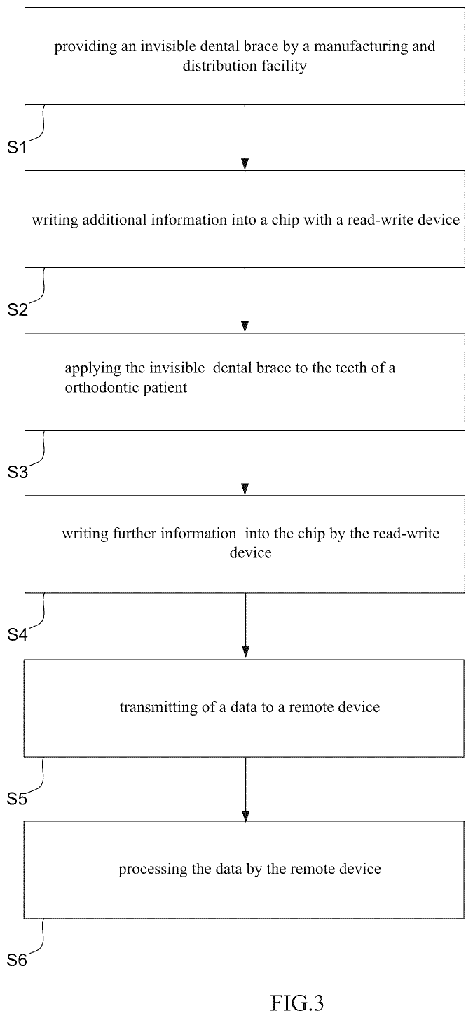

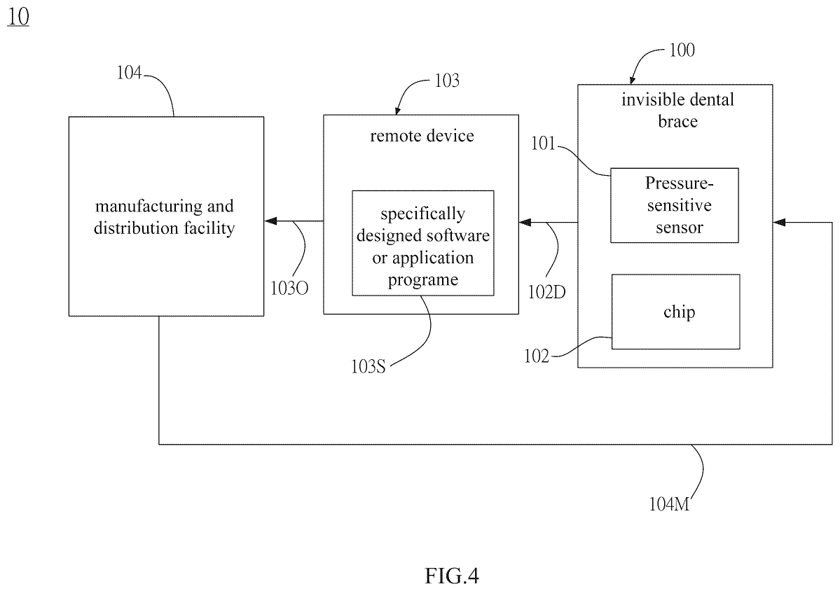

FIG. 1 is an illustrative diagram that shows an invisible dental brace 100 containing a pressure-sensitive sensor 101 according to the present invention. FIG. 2 is an illustrative diagram of a system 10 for the management and distribution of invisible dental braces 100 containing pressure-sensitive sensors 101 according to the present invention in which data 102D is transmitted from a chip 102 of the invisible dental brace 100 to a remote device 103. FIG. 3 is a flowchart showing the steps for the management and distribution of invisible dental braces 100 containing pressure-sensitive sensors 101. FIG. 4 is a block diagram of a system 10 for the management and distribution of invisible dental braces 100 containing pressure-sensitive sensors 101, comprising a manufacturing and distribution facility 104, an invisible dental brace 100 containing pressure-sensitive sensors 101, and a remote device 10.

According to FIG. 1, an invisible dental brace 100 is arranged with a pressure-sensitive sensor 101, wherein the pressure-sensitive sensor 101 is configured to generate a sensor signal related to the wearing time of the invisible dental brace 100 by an orthodontic patient and a pressure applied by the teeth 200 of an orthodontic patient to the invisible dental brace 100. In addition, a chip 102 is also arranged on the invisible dental brace 100 and configured to store the sensor signal and other information including the identification number of the invisible dental brace 100, the name of the orthodontic patient wearing the invisible dental brace 100, an initial time of wearing, and special conditions that occur during applying the invisible dental brace 100 to the teeth 200 of the orthodontic patient.

FIG. 2 is a system 10 for the management and distribution of invisible dental braces 100 containing pressure-sensitive sensors 101, in which data 102D stored in chip 102 is transmitted to a remote device 103 and processed therein with a specifically designed software or application program 103S to produce an understandable and visual chart and 3D images representing the current teeth configuration of the orthodontic patient for the dentist to adjust the prescribed plan of orthodontic treatment accordingly, wherein the data 102D including the sensor signal, identification number of the invisible dental brace, name of the orthodontic patient wearing the invisible dental brace, an initial time of wearing, and special conditions that occur during applying the invisible dental brace to the teeth of the orthodontic patient. When a modified prescribed plan of orthodontic treatment is done based on the data, the dentist delivers an order 103O of a new invisible dental brace by the specifically designed software or application program 103S from a manufacturing and distribution facility 104. After the manufacturing and distribution facility 104 receives the order 103O, the manufacturing and distribution facility 104 initiates a manufacture 104M of the new invisible dental brace.

According to FIG. 3 and FIG. 4, a system and method for the management and distribution of invisible dental braces 100 containing pressure-sensitive sensors 101 according to the present invention comprises the following steps:
S1: providing an invisible dental brace 100 by a manufacturing and distribution facility 104 to a dentist based on a 3D image generated from an intraoral scanning of an orthodontic patient, wherein at least one pressure-sensitive sensor 101 and a chip 102 are fixedly secured onto the invisible dental brace 100, wherein said at least one pressure-sensitive sensor 101 is configured to generate a sensor signal related to wearing time of the invisible dental brace 100 and a pressure applied by teeth 200 of the orthodontic patient, wherein an identification number regarding the invisible dental brace 100 is written into the chip 102;
S2: writing additional information, including the name of the orthodontic patient wearing the invisible dental brace 100 and an initial time of wearing, into the chip 102 by a read-write device provided by the manufacturing and distribution facility 104 to the dentist;
S3: applying the invisible dental brace 100 to the teeth 200 of the orthodontic patient by the dentist;
S4: writing further information into the chip 102 by the read-write device, including special conditions that occur during applying the invisible dental brace 100 to the teeth 200 of the orthodontic patient;
S5: transmitting of a data 102D, including the sensor signal, identification number, additional information, and further information through wire or a wireless communication technology to a remote device 103 during the usage of the invisible dental brace 100 by the patient; and
S6: processing the data 102D by a specifically designed software or application program 103S of the remote device 103 for the dentist to modify the prescribed plan of orthodontic treatment in a real-time manner based on the data 102D, and based on a modified prescribed plan of orthodontic treatment, an order 103O of a new invisible dental brace is delivered to the manufacturing and distribution facility 104 to initiate a manufacture 104M of the new invisible dental brace.

In one embodiment, the chip 102 contains a memory, and the memory can be an EEPROM (Electrically-Erasable Programmable Read-Only Memory) or a flash memory for storing information. The memory can be divided into two parts—one read-only part for storing encrypted read-only information and one read-write part. The read-only part of the memory is for storing information such as a discrete identification number assigned to the invisible dental brace 100. The read-write part of chip 102 is for storing the data 102D, including sensor signals, additional information, and further information.

In one embodiment, the data 102D can be transmitted to a remote device 103 through a wire or a wireless communication technology. Further, chip 102 contains a wireless communication circuit, and the data 102D can be transmitted to a remote device 103 through any existing wireless communication technologies including Wi-Fi, Bluetooth, WiMax, and cellular network.

In one embodiment, said at least one pressure-sensitive sensor 101 is selected from the following: a force-sensitive film, a pressure-sensitive film, a resistive film, a capacitive film or a piezoresistive pressure sensor.

In one embodiment, the remote device 103 can be any computing device or system, such as a personal computer, laptop, tablet, mobile device, wearable device, etc.

In one embodiment, the remote device 103 is installed with a specifically designed software or application program 103S that can process the data 102D into an understandable and visual chart and 3D images representing the current teeth configuration of the orthodontic patient for the dentist to adjust the prescribed plan of orthodontic treatment accordingly. Furthermore, the specifically designed software or application program 103S is updatable through the service of the manufacturing and distribution facility 104 to provide an up-to-date service.

In a nutshell, the above descriptions have thoroughly introduced the system and method for the management and distribution of invisible dental braces containing pressure-sensitive sensors according to the present invention. The above descriptions are made on embodiments of the present invention; however, the embodiments are not intended to limit the scope of the present invention, and all equivalent implementations or alterations within the spirit of the present invention still fall within the scope of the present invention.

For some scenarios involving the invention of the system and method for the management and distribution of invisible dental braces with pressure-sensitive sensors could unfold in various ways.

Real-time Treatment Adjustments: A patient wears the invisible dental brace, and the pressure-sensitive sensors monitor the forces applied to the teeth.

Special conditions, such as increased pressure or irregularities during brace application, are recorded in real-time.

The data, including sensor signals and additional information, is transmitted to a remote device, allowing the orthodontist to monitor and adjust the treatment plan immediately.

In the embodiment, the invisible dental braces comprise embedded pressure-sensitive sensors and chips, and remote monitoring device (smartphone, computer, tablet) for the orthodontist.

The operation could be: Patient wears the invisible dental brace, and sensors monitor pressure on the teeth. Sensor signals indicating forces applied are generated in real-time. Special conditions during brace application, recorded by the sensors, are transmitted. Data is sent to the remote device of the orthodontist. Orthodontist analyzes the data and modifies the treatment plan if necessary.

Remote Monitoring and Consultation: Patients living in remote areas or unable to visit the orthodontist frequently benefit from the system.

The orthodontist receives regular updates on the patient's progress through transmitted data.

Remote consultations are facilitated, and treatment plans can be modified without requiring the patient to be physically present.

In the embodiment, invisible dental braces comprise embedded sensors, and remote device for the patient (smartphone, computer).

The operation could be: Patient wears the braces, and sensor data is collected. Periodic transmissions of data to the remote device. Orthodontist receives data remotely and conducts virtual consultations. Treatment adjustments are made based on the remote consultation.

Enhanced Patient Engagement: Patients become more engaged in their orthodontic treatment with real-time feedback.

The system encourages adherence to the treatment plan as patients can see the effects of their actions on the sensors.

Gamification elements may be incorporated to make the treatment process more interactive and enjoyable.

In the embodiment, invisible dental braces comprise sensors and chips. Patient's device for monitoring (smartphone, computer).

The operation could be: Sensors record pressure data during daily activities. Patients receive real-time feedback on their device. Gamified elements may include progress tracking, achievements, or challenges. Patients can visually see the impact of their actions on the treatment.

Data-Driven Orthodontics: Over time, a large dataset is accumulated, providing valuable insights into the effectiveness of different treatment approaches.

Machine learning algorithms could be implemented to analyze patterns and suggest optimized treatment plans based on historical data.

In the embodiment, invisible dental braces comprise sensors and cloud database.

The operation could be: Continuous collection of pressure data from multiple patients. Aggregation of anonymized data in the cloud. Implementation of machine learning algorithms for pattern recognition. Algorithms suggest optimized treatment plans based on historical data.

Improved Treatment Outcomes: With real-time monitoring, adjustments can be made promptly, potentially reducing the overall duration of orthodontic treatment.

Patients experience fewer complications, and treatment outcomes are optimized.

In the embodiment, invisible dental braces comprise embedded sensors, and remote monitoring device for the orthodontist.

The operation could be: Regular transmission of treatment progress data. Orthodontist analyzes the data and makes timely adjustments. Reduced chances of complications lead to improved treatment outcomes.

Customized Orthodontic Care: The system allows for a more personalized approach to orthodontic care.

Individual patient data, including their responses to pressure and unique conditions, contributes to tailoring treatment plans for better results.

In the embodiment, invisible dental braces with sensors.

The operation could be: Unique patient responses to pressure are recorded. Treatment plans are tailored based on individual patient data. Personalized adjustments lead to more effective orthodontic care.

Integration with Other Health Metrics: The system may integrate with other health monitoring devices, providing a holistic view of a patient's well-being during orthodontic treatment.

Health-related data, such as stress levels or sleep patterns, could be considered in modifying treatment plans.

In the embodiment, invisible dental braces with sensors, and other health monitoring devices (wearables, sensors).

The operation could be: Integration of data from dental sensors with health metrics. Comprehensive health data provides a holistic view. Orthodontic treatment plans consider broader well-being factors.

Global Collaboration and Research: Aggregated and anonymized data from patients worldwide contribute to orthodontic research and advancements.

Global collaboration among orthodontists becomes easier, leading to the development of standardized best practices.

In the embodiment, invisible dental braces with sensors, and global cloud database.

The operation could be: Aggregated, anonymized data from patients worldwide. Orthodontists globally contribute to a shared pool of data. Standardized best practices are developed through collaborative research.

For another embodiment, a system of invisible dental braces equipped with pressure-sensitive sensors, comprising: a set of invisible dental braces, each brace comprising a pressure-sensitive sensor for monitoring the pressure exerted on the teeth; a data collection unit for collecting data from the pressure-sensitive sensors; a data analysis unit for analyzing the collected data to determine the effectiveness of the orthodontic treatment; a communication unit for transmitting the analyzed data to a remote server; a remote server for storing and managing the analyzed data; a user interface for accessing and displaying the analyzed data to the user.

Wherein the pressure-sensitive sensors are embedded within the invisible dental braces. The pressure-sensitive sensors are configured to measure the pressure exerted on individual teeth. The data collection unit further comprises a wireless receiver for wirelessly receiving data from the pressure-sensitive sensors. Wherein the data analysis unit is configured to generate reports indicating the progress of the orthodontic treatment. Wherein the user interface is accessible via a mobile application.

For another embodiment, a system of invisible dental braces comprising: a set of invisible dental braces, each brace comprising pressure-sensitive sensors embedded within; a chip for recording wear time and tooth pressure data; a secure online platform for remote access of recorded data; a database for storing additional patient data including dental impressions and treatment plans.

Wherein the pressure-sensitive sensors are capable of continuously monitoring the forces exerted on the teeth, the chip is capable of wirelessly transmitting the recorded wear time and tooth pressure data to the secure online platform.

Wherein the secure online platform further comprises a user interface for displaying the recorded wear time and tooth pressure data.

The database further comprises a treatment progress tracker for monitoring the progress of orthodontic treatment. The pressure-sensitive sensors are capable of detecting abnormal forces exerted on the teeth and generating alerts to the orthodontist.

Wherein the chip further comprises a memory for storing the recorded wear time and tooth pressure data, the secure online platform further comprises a communication module for facilitating communication between the orthodontist and the patient.

Wherein the pressure-sensitive sensors are capable of detecting the alignment of the teeth and generating alignment data for analysis by the orthodontist, the chip further comprises a power source for powering the pressure-sensitive sensors and the recording of wear time and tooth pressure data.

What is claimed is:

1. A method for management and distribution of invisible dental brace containing pressure-sensitive sensors comprising the steps of:
providing an invisible dental brace by a manufacturing and distribution facility to a dentist, wherein at least one pressure-sensitive sensor and a chip are fixedly secured onto the invisible dental brace, wherein said at least one pressure-sensitive sensor is configured to generate a sensor signal related to wearing time of the invisible dental brace and a pressure applied by teeth of an orthodontic patient, wherein an identification number regarding the invisible dental brace is written into the chip;
writing additional information, including the name of the orthodontic patient wearing the invisible dental brace and an initial time of wearing, into the chip by a read-write device provided by the manufacturing and distribution facility to the dentist;
applying the invisible dental brace to the teeth of the orthodontic patient by the dentist;
writing further information into the chip by the read-write device, including special conditions that occur during applying the invisible dental brace to the teeth of the orthodontic patient;
transmitting of a data, including the sensor signal, identification number, additional information, and further information through wire or a wireless communication technology to a remote device during the usage of the invisible dental brace by the patient; and
processing the data by a specifically designed software or application program of the remote device for the dentist to modify the prescribed plan of orthodontic treatment in a real-time manner based on the data, and based on a modified prescribed plan of orthodontic treatment, an order of a new invisible dental brace is delivered to the manufacturing and distribution facility to initiate a manufacture of the new invisible dental brace.

2. A system of invisible dental braces equipped with pressure-sensitive sensors, comprising:
a set of invisible dental braces, each brace comprising a pressure-sensitive sensor for monitoring the pressure exerted on the teeth;
a data collection unit for collecting data from the pressure-sensitive sensors;
a data analysis unit for analyzing the collected data to determine the effectiveness of the orthodontic treatment;
a communication unit for transmitting the analyzed data to a remote server;
a remote server for storing and managing the analyzed data;
a user interface for accessing and displaying the analyzed data to the user.

3. The system of claim 1, wherein the pressure-sensitive sensors are embedded within the invisible dental braces.

4. The system of claim 1, wherein the pressure-sensitive sensors are configured to measure the pressure exerted on individual teeth.

5. The system of claim 1, wherein the data collection unit further comprises a wireless receiver for wirelessly receiving data from the pressure-sensitive sensors.

6. The system of claim 1, wherein the data analysis unit is configured to generate reports indicating the progress of the orthodontic treatment.

7. The system of claim 1, wherein the user interface is accessible via a mobile application.

8. A system of invisible dental braces comprising:
a set of invisible dental braces, each brace comprising pressure-sensitive sensors embedded within;
a chip for recording wear time and tooth pressure data;
a secure online platform for remote access of recorded data;
a database for storing additional patient data including dental impressions and treatment plans.

9. The system of claim 8, wherein the pressure-sensitive sensors are capable of continuously monitoring the forces exerted on the teeth.

10. The system of claim 8, wherein the chip is capable of wirelessly transmitting the recorded wear time and tooth pressure data to the secure online platform.

11. The system of claim 8, wherein the secure online platform further comprises a user interface for displaying the recorded wear time and tooth pressure data.

12. The system of claim 8, wherein the database further comprises a treatment progress tracker for monitoring the progress of orthodontic treatment.

13. The system of claim 8, wherein the pressure-sensitive sensors are capable of detecting abnormal forces exerted on the teeth and generating alerts to the orthodontist.

14. The system of claim 8, wherein the chip further comprises a memory for storing the recorded wear time and tooth pressure data.

15. The system of claim 8, wherein the secure online platform further comprises a communication module for facilitating communication between the orthodontist and the patient.

16. The system of claim 8, wherein the pressure-sensitive sensors are capable of detecting the alignment of the teeth and generating alignment data for analysis by the orthodontist.

17. The system of claim 8, wherein the chip further comprises a power source for powering the pressure-sensitive sensors and the recording of wear time and tooth pressure data.
